(19) United States
(12) Patent Application Publication
(10) Pub. No.: US 2015/0250887 A1
(43) Pub. Date: Sep. 10, 2015

(54) METHOD OF PREPARING RESIN TINCTURES

(71) Applicant: JALEVA PHARMACEUTICALS, LLC, San Diego, CA (US)

(72) Inventors: Alex Battaglia, La Jolla, CA (US); James Roberts, Prestatyn (GB); Mansoor Ahsan, Barrowford (GB)

(21) Appl. No.: 14/434,857

(22) PCT Filed: Oct. 11, 2013
(86) PCT No.: PCT/US2013/064556

§ 371 Date: Apr. 10, 2015

Related U.S. Application Data
(60) Provisional application No. 61/713,238, filed on Oct. 12, 2012.
(60) Provisional application No. 61/713,290, filed on Oct. 12, 2012.

(51) Int. Cl. A61K 47/30; A61K 47/10; A61K 31/196; A61K 31/203; A61K 31/155; A61K 31/519

(52) U.S. Cl. CPC A61K 47/30; A61K 31/155; A61K 31/519; A61K 31/196; A61K 31/203; A61K 47/10

(57) ABSTRACT

Described herein are methods for making a pharmaceutical composition for topical, transdermal or transmucosal delivery of a pharmaceutically active agent comprising combining (i) a first composition comprising or consisting of a pharmaceutically active agent and, optionally, a first volatile solvent and/or water, and (ii) a second composition comprising a resin, a second volatile solvent and 10-40% v/v water. Compositions made by these methods also are described, as are compositions comprising a pharmaceutically active agent, a volatile solvent, a resin and 10-40% (w/v) water, and containers containing such compositions or components thereof.

### FIELD

Described herein are pharmaceutical compositions for topical, transdermal or transmucosal use, containers containing such compositions or components thereof, methods for making such compositions, and methods of using such compositions to effect topical, transdermal or transmucosal delivery of the active agent.

### BACKGROUND

Tinctures can be produced by dissolving a pharmaceutically active agent in alcohol. However, when producing such tinctures, many pharmaceutically active agents (such as hydrophilic active agents) are insoluble or poorly soluble or become unstable when they are mixed in an alcoholic solution that also includes a resin, such as benzoin or mastic gum. For example, some pharmaceutically active agents may (1) fail to dissolve, (2) precipitate, or (3) break down over time once placed into an alcoholic solution that also includes a resin. Surfactants have been added to such tinctures in an attempt to improve the stability of the active agents, but stability problems may remain. Moreover, surfactants may not be acceptable or desirable for all pharmaceutical uses.

Chlorhexidine is an active agent with antibacterial activity that generally is effective against both Gram-positive and Gram-negative bacteria. Two salt forms have been disclosed for use in pharmaceutical compositions: chlorhexidine acetate and chlorhexidine gluconate. Although chlorhexidine acetate is soluble in an alcoholic solution with resin (e.g., in a tincture), it also acts as a strong skin irritant. See Contact Dermatitis, 24(1): 145-9 (1991). While chlorhexidine gluconate does not exhibit as strong irritant properties, it can be difficult to prepare a stable alcoholic solution (tincture) that includes chlorhexidine gluconate and a resin.

### BRIEF DESCRIPTION OF THE FIGURES

FIG. 1 shows the level of pain (analogue pain scale score) over time after topical treatment with a diclofenac resin composition as described herein versus a placebo, before treatment, 20 minutes after treatment and 60 minutes after treatment.

FIG. 2 shows the accumulated in vitro diffusion of diclofenac across human skin after application of a diclofenac resin composition as described herein (▴) and Voltaren® diclorenac gel (♦) (peak area, mAU v. time (min.)).

FIG. 3 shows the accumulated in vitro diffusion of calcipotriene across human skin after application of a calcipotriene resin composition as described herein (▪) and Dovonex® calcipotriene gel (♦) (peak area, mAU v. time (min.)).

FIG. 4 shows the accumulated in vitro diffusion of retin-A (tretinoin) across human skin after transdermal application of a retin-A resin composition as described herein (▪) and a commercial tretinoin gel (♦)(peak area, mAU v. time (min.)).

### SUMMARY

One embodiment relates to a method of preparing a chlorhexidine gluconate pharmaceutical composition for topical delivery, comprising combining (i) a first composition comprising chlorhexidine gluconate, a first volatile solvent, and 1-40% (v/v) water; and (ii) a second composition comprising a resin, a second volatile solvent, and 1-40% (v/v) water, wherein the final amount of water in the chlorhexidine gluconate pharmaceutical composition is up to 40% (v/v). Another more general embodiment relates to a method of preparing a pharmaceutical composition for topical, transdermal or transmucosal delivery of a pharmaceutically active agent, comprising combining (i) a first composition comprising or consisting of a pharmaceutically active agent and optionally comprising a first volatile solvent and/or water and (ii) a second composition comprising a resin, a second volatile solvent, and water. In any of these embodiments, the resin may be benzoin or mastic gum.

In some embodiments, at least one of the first composition and the second composition is filtered using a syringe filter or a large scale commercial filter. In specific embodiments, the syringe filter is a 5-micron or a 0.45-micron nylon syringe filter.

In some embodiments, the first and/or the second volatile solvents are independently selected from the group consisting of alcohols, ketones and ethers. In any of these embodiments, the first and/or the second volatile solvents are independently selected from the group consisting of ethyl acetate, n-propyl acetate, methanol, ethanol, propanol, isopropanol, isopropyl alcohol, acetone and dimethyl ether. In specific embodiments, at least one of the first and second volatile solvents is isopropyl alcohol.

Some embodiments further comprise adding to the pharmaceutical composition one or more additional components selected from the group consisting of dyes, fragrances, flavors, penetration enhancers, and pharmaceutically acceptable carriers. In some embodiments, the first composition further comprises one or more additional components selected from the group consisting of dyes, fragrances, flavors, penetration enhancers, and pharmaceutically acceptable carriers. In some embodiments, the second composition further comprises one or more additional components selected from the group consisting of dyes, fragrances, flavors, penetration enhancers, and pharmaceutically acceptable carriers.

Some embodiments comprise preparing the second composition by combining the resin, volatile solvent and water, and allowing the composition to settle. Other specific embodiments further comprise filtering the second composition after allowing the composition to settle.

In some embodiments, the pH of the second composition is adjusted before combining with the first composition, such as may be appropriate to promote a log D of the pharmaceutically active agent that is suitable or advantageous for transdermal or transmucosal delivery.

Some embodiments relate to pharmaceutical compositions made by one or more of the methods described herein.

Some embodiments relate to a composition comprising a pharmaceutically active agent, volatile solvent, a resin, and 10-40% w/v water. In specific embodiments, the resin is benzoin or mastic gum.

In specific embodiments, the pharmaceutically active agent is selected from the group consisting of nicotine, scopolamine, lidocaine, benzocaine, ketorolac, ibuprofen, ketoprofen, flurbiprofen, naproxen, astemizole, terfenadine, cimetidine, testosterone, retin-A. In other specific embodiments, the pharmaceutically active agent is selected from the group consisting of ondansetron, granisetron, zolmitriptan, dihydroergotamine, sumatriptan, rizatriptan, fentanyl, cocaine, alprazolam, clonazepam, lorazepam, diazepam, estazolam, apomorphine, risperidone, buprenorphine, naloxone, flumazenil, tadalafil, vardenafil, sildenafil, sildenafil mesylate, dolasetron, palonsetron, triazolam, naratriptan, diclofenac, etololac, meclofenamate, indocin, meloxicam, nabumetone, oxaprozin, prioxicam, sulindac, tolmetin, celecoxib, loratadine, desloratidine, cetirizine, morphine, hydromorphine, levorphanol, meperidine, oxycodone, oxymorphone, propanolol, calcitriol, and methylphenidate.

In specific embodiments, the volatile solvent is selected from the group consisting of alcohols, ketones and ethers, and mixtures thereof, such as ethyl acetate, n-propyl acetate, methanol, ethanol, propanol, isopropanol, isopropyl alcohol, acetone and dimethyl ether, and mixtures thereof, such as isopropyl alcohol.

Some embodiments relate to a container comprising: (i) a first chamber comprising a first composition comprising or consisting of a pharmaceutically active agent and optionally a first volatile solvent and/or water and (ii) a second chamber comprising a second composition comprising a resin, a second volatile solvent, and water. In some embodiments, the container is an applicator.

Some embodiments relate to a method of topically, transdermally or transmucosally administering a pharmaceutically active agent to an individual, comprising applying any pharmaceutical composition as described herein to the skin, nail or mucosa of the individual.

### DETAILED DESCRIPTION

Technical and scientific terms used herein have the meanings commonly understood by one of ordinary skill in the art to which the present invention pertains, unless otherwise defined. Reference is made herein to various methodologies known to those of ordinary skill in the art. Publications and other materials setting forth such known methodologies to which reference is made are incorporated herein by reference in their entireties as though set forth in full. Any suitable materials and/or methods known to those of ordinary skill in the art can be utilized in carrying out the present invention. However, specific materials and methods are described. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only. Likewise, singular forms of terms designate both the singular and plural, unless expressly stated to designate the singular only. For example, “active agent” means “active agent” or “active agents.”

The term “about” in connection with numerical values and ranges means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10%, plus or minus 5%, or plus or minus 1%, of the particular term.

The phrase “substantially free” as used herein generally means that the described composition (e.g., pharmaceutical composition, etc.) comprises less than about 5%, less than about 3%, or less than about 1%, by weight, based on the total weight of the composition at issue, of the excluded component.

As used herein “subject” or “patient” denotes any animal in need of treatment with a pharmaceutically active agent. For example, a subject may be suffering from or at risk of developing a condition that can be treated or prevented with a pharmaceutically active agent, or may be administering a pharmaceutically active agent for health maintenance purposes. As used herein “subject” or “patient” includes humans.

As used herein, the phrases “therapeutically effective amount” and “therapeutic level” mean that drug dosage or plasma concentration in a subject, respectively, that provides the specific pharmacological response for which the drug is administered in a subject in need of such treatment. It is emphasized that a therapeutically effective amount or therapeutic level of a drug will not always be effective in treating the conditions/diseases described herein, even though such dosage is deemed to be a therapeutically effective amount by those of skill in the art. For convenience only, exemplary dosages, drug delivery amounts, therapeutically effective amounts and therapeutic levels are provided below with reference to adult human subject. Those skilled in the art can adjust such amounts in accordance with standard practices as needed to treat a specific subject and/or condition/disease.

As used herein, the term “skin” refers to the membranous tissue forming the external covering or integument of an animal and consisting in vertebrates of the epidermis and dermis.

As used herein, the term “nail” refers to a substructure, composed mainly of the protein keratin, of the outer layer of the skin. As such, “nail” includes both “fingernails” and “toenails” of an animal. The “nail bed” is the skin on the top of which the nail grows.

As used herein, the term “mucosa” refers to mucous membrane epithelium, including oral mucous membranes including buccal mucous membranes.

Described herein are methods for making pharmaceutical compositions comprising a resin for use on the skin, nail or oral mucosa, as well as to pharmaceutical compositions made by the methods, and compositions for topical, transdermal, or transmucosal administration. In particular, as described in more detail below, described herein are methods for preparing pharmaceutical compositions by combining (a) a first composition comprising or consisting of at least one pharmaceutically active agent, optionally further comprising a first volatile solvent, and/or water, and (b) a second composition comprising a resin, a second volatile solvent and/or water, and/or, optionally, an anionic dye, to obtain a pharmaceutical composition. It was surprisingly found that pharmaceutical compositions prepared by this method exhibit increased stability. For example, in specific embodiments, the pharmaceutical compositions prepared by this method exhibit good dissolution of the pharmaceutically active agent. Additionally or alternatively, in some embodiments, the pharmaceutically active agent does not precipitate out of solution or break down over time. Thus, compositions made by these methods are convenient and effective for the transdermal or transmucosal delivery of pharmaceutically active agents.

In accordance with some embodiments, the pharmaceutical compositions described herein provide improved delivery of pharmaceutically active agents that are less soluble in organic solvents (such as alcohols), and other pharmaceutically active agents that heretofore have not been able to be stably formulated in resin tinctures. For example, many pharmaceutically active agents that are applied topically for the treatment dermatologic disorders can be formulated as described herein, and can be applied to specific sites affected by, for example, a skin disorder or a surgical site.

In accordance with some embodiments, the pharmaceutical compositions described herein provide improved delivery of chlorhexidine gluconate. The pharmaceutical compositions described herein also allow a more specifically directed application of chlorhexidine gluconate to specifically selected application sites affected by, for example, a skin disorder or a surgical site.

In specific embodiments, the compositions described herein form a film on the site of application, after application and evaporation of the volatile solvent(s). These embodiments may achieve extended retention of the pharmaceutically active agent on the site of application because the film is resistant to water and abrasion.

In specific embodiments where the compositions contain chlorhexidine gluconate, the compositions exhibit stability with regard to the chlorhexidine gluconate, and do not suffer from unacceptable instability problems when the composition is formulated with a dye, such as an anionic dye, as may be observed with other chlorhexidine gluconate resin formulations. These embodiments also can provide adhesion to a medical device, such as to a surgical drape. These embodiments may also achieve extended retention of the chlorhexidine gluconate on the site of application because the film is resistant to water and abrasion.

The sustained application of the foregoing embodiments is beneficial in many situations, such as in the use of compositions comprising anti-infection agents on surgical sites. Additionally, many dermatological conditions are exacerbated by moisture, so the water-repellent qualities of the film offer particular advantages in that context by protecting the application site from moisture that may cause further damage. Nevertheless, the films can be conveniently and easily removed, for example with alcohol or washing or rinsing, when desired. Thus, the pharmaceutical compositions described herein can be formulated for direct application to a site needing treatment (such as a lesion or surgical site), and can be left in place for an extended period of time, without requiring a conventional adhesive bandage.

The skilled artisan will understand that these advantages are not limited to the context of the treatment of these specific conditions, but can be beneficial in the context of other conditions and disorders as well.

Methods of Preparing Pharmaceutical Compositions

Described herein are methods for preparing pharmaceutical compositions comprising combining (a) a first composition comprising or consisting of a pharmaceutically active agent and optionally further comprising a first volatile solvent and/or water and (b) a second composition comprising a resin, a second volatile solvent, and water. In some embodiments, the methods comprise preparing chlorhexidine gluconate pharmaceutical compositions comprising (a) separately preparing (1) a first mixture comprising chlorhexidine gluconate, a first volatile solvent, and water and (2) a second mixture comprising a resin, a second volatile solvent, and water, and (b) combining the first and second mixtures to obtain a pharmaceutical composition. In some embodiments, the second composition is prepared by methods described herein below. Optionally, one or more other ingredients, such as dyes, fragrances, flavors, penetration enhancers, and pharmaceutically acceptable carriers, can be added to one or both of the first and second compositions, or can be added after the first and second compositions are combined.

In specific embodiments, the relative proportions of the components of the first and second compositions are selected to promote the stability of the pharmaceutically active agent in the pharmaceutical composition.

The relative amounts of the components can be varied from and within the ranges outlined below in order to obtain a composition with desired pharmaceutical strength, desired pharmacokinetic properties, and/or desired physical properties, which may depend on the site of application (e.g., skin versus nail versus mucosa). For example, the relative amounts may affect the rate of release of the pharmaceutically active agent, the tackiness of the film, and/or the thickness or area of application. For example, a composition formulated for application to the face may have a lower relative amount of resin to provide a less sticky composition and a thinner film upon application and evaporation of the volatile solvent(s).

In some embodiments, at least one of the first composition and the second composition is filtered prior to combining with the other composition. In specific embodiments, the first composition is filtered prior to combining with the second composition. In other specific embodiments, the second composition is filtered prior to combining with the first composition. In other specific embodiments, each of the first composition and the second composition is filtered prior to combining with the other composition. Such filtering my be effecting by any suitable means. In some embodiments, filtering is affected using a syringe filter or a large scale commercial filter. In specific embodiments, the syringe filter is a 5-micron or a 0.45-micron nylon syringe filter.

The pharmaceutical compositions can be prepared as a sticky slurry or solution, which can be applied to a site on the skin, nail or a mucosal membrane, such as the buccal mucosal membrane. The consistency of the pharmaceutical composition can be varied by, for example, adjusting the relative amount of volatile solvent and resin. For areas where evaporation of the volatile solvent may be slower, such as mucosal membrane such as the gums, a composition with less volatile solvent may be advantageous. On the other hand, for areas that are hard to reach, such as between the toes, a composition with more volatile solvent may be advantageous. Still, for treatment of more severe lesions, such as due to athlete's foot infection, for example, a more viscous composition (with less volatile solvent) may be advantageous.

In some embodiments, the second mixture is combined with a dye before combining with the first mixture. In specific embodiments, the second mixture is combined with a dye and then allowed to settle before combining with the first mixture. In some embodiments, the settling time is from about 1 day to about 4 months, including about 1 month, typically at room temperature. In other embodiments, the settling time is at lower temperatures, such as at 4° C.

In some embodiments, prior to combining with the first mixture, the pH of the second mixture is adjusted, such as to a pH that supports the antimicrobial activity of chlorhexidine gluconate, such as a pH of from about 5.5 to about 7.0, including from about 6.0 to about 7.0. In embodiments where the second mixture is combined with a dye and then allowed to settle, the pH may be adjusted after settling and before combining with the first mixture.

In some embodiments, the pH of the pharmaceutical composition is adjusted, such as to a pH that supports the antimicrobial activity of chlorhexidine gluconate, such as a pH of from about 5.5 to about 7.0, including from about 6.0 to about 7.0.

Pharmaceutically Active Agents

As the pharmaceutically active agent, any pharmaceutically active agent can be used. In specific embodiments, the pharmaceutically active agent is topically active or capable of transdermal or transmucosal delivery. In specific embodiments, the pharmaceutically active agent is chlorhexidine glucoate. In specific embodiments, the pharmaceutically active agent is hydrophilic. In specific embodiments, the pharmaceutically active agent is insoluble or poorly soluble or becomes unstable when mixed in an alcoholic solution that also includes a resin, such as benzoin or mastic gum. For example, such pharmaceutically active agents may (1) fail to dissolve, (2) precipitate, or (3) break down over time once placed into an alcoholic solution that also includes a resin. For example, the pharmaceutically active agent may be one that heretofore has not been capable of being stably formulated in a resin tincture.

In some embodiments, more than one pharmaceutically active agent can be included in the first composition. In specific embodiments, two pharmaceutical agents are included in the first composition. In specific embodiments, three or more pharmaceutical agents are included in the first composition.

In the description that follows, specific pharmaceutically active agent are disclosed. It is to be understood that pharmaceutically acceptable salts, esters, and other pharmaceutically acceptable forms of the listed active agents (including prodrugs) are included in the disclosure.

In some embodiments, the pharmaceutically active agent is chlorhexidine gluconate. In general, the chlorhexidine gluconate may be present at a concentration typically used in a topical formulation. In some embodiments, the chlorhexidine gluconate constitutes less than about 25% of the first mixture, such as from about 0.1% (w/v) to about 25% (w/v), or from about 0.1% (w/v) to about 20% (w/v), or from about 0.1% (w/v) to about 15% (w/v), or from about 0.1% (w/v) to about 10% (w/v), or from about 0.1% (w/v) to about 5% (w/v), or from about 0.1% (w/v) to about 1% (w/v), or from about 0.5% (w/v) to about 25% (w/v), or from about 0.5% (w/v) to about 20% (w/v), or from about 0.5% (w/v) to about 15% (w/v), or from about 0.5% (w/v) to about 10% (w/v), or from about 0.5% (w/v) to about 5% (w/v), or from about 0.5% (w/v) to about 1% (w/v), or from about 1% (w/v) to about 25% (w/v), or from about 1% (w/v) to about 20% (w/v), or from about 1% (w/v) to about 15% (w/v), or from about 1% (w/v) to about 10% (w/v), or from about 1% (w/v) to about 5% (w/v), or from about 5% (w/v) to about 25% (w/v), or from about 5% (w/v) to about 20% (w/v), or from about 5% (w/v) to about 15% (w/v), or from about 5% (w/v) to about 10% (w/v), or from about 10% (w/v) to about 25% (w/v), or from about 10% (w/v) to about 20% (w/v), or from about 10% (w/v) to about 15% (w/v), or from about 15% (w/v) to about 25% (w/v), or from about 15% (w/v) to about 20% (w/v), or from about 20% (w/v) to about 25% (w/v), including from 0.1-25% (w/v), 0.1-20% (w/v), 0.1-15% (w/v), 0.1-10% (w/v), 0.1-5% (w/v), 0.1-1% (w/v), 0.5-25% (w/v), 0.5-20% (w/v), 0.5-15% (w/v), 0.5-10% (w/v), 0.5-5% (w/v), 0.5-1% (w/v), 1-25% (w/v), 1-20% (w/v), 1-15% (w/v), 1-10% (w/v), 1-5% (w/v), 5-25% (w/v), 5-20% (w/v), 5-15% (w/v), 5-10% (w/v), 10-25% (w/v), 10-20% (w/v), 10-15% (w/v), 15-25% (w/v), 15-20% (w/v) and 20-25% (w/v), including 0.1% (w/v), 0.5% (w/v), 1% (w/v), 2% (w/v), 3% (w/v), 4% (w/v), 5% (w/v), 10% (w/v), 15% (w/v), 20% (w/v) or 25% (w/v).

In specific embodiments, the amount of chlorhexidine gluconate in the first mixture is selected to provide a final composition comprising chlorhexidine gluconate in a range of 0.1% to 5% (w/v).

Because the adherent properties of the compositions may provide extended drug exposure and/or controlled drug delivery, in some embodiments a lower concentration can be used while still achieving the desired pharmaceutical effect. In any event, the amount of chlorhexidine gluconate can be determined by the skilled artisan. Generally, the amount used will be within the range of +/−0.25% of the indicated concentration, such as within +/−10% of the indicated concentrations.

In some embodiments, the pharmaceutically active agent is selected from the following classes and subclasses. In specific embodiments, the pharmaceutically active agent is more water soluble than alcohol soluble. For example, pharmaceutically active agent may be one or more selected from the following, and in particular may be one or more selected from the following which is more water soluble than alcohol soluble: analgesics (e.g., antirheumatics, muscle relaxants, opiods, non-opiods, NSAIDS), anesthetics (e.g., sedatives, local anesthetics, neuromuscular blockers); antimicrobials (e.g., aminoglycosides, antifungals, antimalarials, antimycobacterials, antiparasitics, antivirals, carbapenems, cephalosporins, ketolids, macrolides, penicillins, quinolones, sulfonamides, tetracyclines), cardiovascular medications (e.g., ACE inhibitors, aldosterone inhibitors, angiotensin blockers, antiadrenergic agents, antidysrhythmics, antihyperlipidemics, antihypertensives, antiplatelet drugs, beta blockers, calcium channel blockers, diuretics, nitrates, pressors/inotropes, thrombolytics, volume expanders), dermatologic medications (e.g., acne, actinic keratosis, antibacterials, antifungals, antiparasitics, antipsoriatics, antivirals, atopic dermatitis, corticosteroids), endocrine/metabolic medications (e.g., androgens, bisphosphonates, corticosteroids, diabetes related, gout related, thyroid agents), ENT medications (e.g. antihistamines, antitussives, decongestants), gastroenterology medications (e.g., anti-diarrheals, antiemetics, antiulcer, laxatives), hematologic anticoagulants, herbal and alternative medications, neurological agents (e.g., anti-Alzheimer's agents, anticonvulsants, migraine medications, multiple sclerosis, myasthenia gravis, parkinsonian agents), OB/GYN agents (e.g., contraceptives, estrogens, GnRH agonists, hormone replacements, labor inducers, ovarian stimulants, progestins, selective estrogen receptor modulators, tocolytics, uterotonics), oncologics (e.g., alkylating agents, antimetabolites, cytoprotective agents, hormones, immunomodulators, mitotic inhibitors, platinum-containing agents, radiopharmaceuticals), psychiatric agents (e.g., antidepressants, antimanic agents, antipsuchotics, anxiolytics, drug dependence, stimulants), pulmonary agents (e.g., beta agonists, inhaled steroids, leukotriene inhibitors), toxicology agents, urology agents (e.g., benign prostate hypertrophy medications, bladder agents, erectile dysfunction medications, nephrolitihiasis agents).

In some embodiments, the pharmaceutically active agent is nicotine, cocaine, buprenorphine, naloxone, flumazenil (used, for example, to treat nicotine or cocaine addiction; opioid addiction; or as a benzodiazepine reversal agent, respectively).

In some embodiments, the pharmaceutically active agent is tadalafil, vardenafil or sildenafil, including sildenafil mesylate (used, for example, to treat erectile dysfunction).

In some embodiments, the pharmaceutically active agent is ondansetron, granisetron, dolasetron or palonsetron (used, for example, to treat or prevent nausea or vomiting).

In some embodiments, the pharmaceutically active agent is a benzodiazepine agent, such as alprazolam (used, for example, to treat anxiety or panic disorders), triazolam (used, for example, to treat insomnia) or clonazepam (used, for example, to treat seizures).

In some embodiments, the pharmaceutically active agent is dihydroergotamine, zolmitriptan, sumatriptan, naratriptan or rizatriptan (used, for example, to treat headaches).

In some embodiments, the pharmaceutically active agent is a non-steroidal anti-inflammatory drug (NSAID), such as ketorolac, ibuprofen, diclofenac, ketoprofen, flurbiprofen, etololac, naproxen, meclofenamate, indocin, meloxicam, nabumetone, oxaprozin, prioxicam, sulindac, tolmetin or celecoxib.

In some embodiments, the pharmaceutically active agent is an anti-histamine compound and/or a steroidal compound, such as histamine H1 and H2 receptor blockers, including astemizole, terfenadine, cimetidine, loratadine, desloratadine and cetirizine (used, for example, for the treatment of allergies and the relief of certain inflammatory dermatological conditions and/or to relieve itching that is often associated with inflammatory lesions).

In some embodiments, the pharmaceutically active agent is fentanyl, morphine, hydromorphine, levorphanol, meperidine, oxycodone or oxymorphone (used, for example, to treat pain).

In some embodiments, the pharmaceutically active agent is apomorphine (used, for example, to treat Parkinson's Disease).

In some embodiments, the pharmaceutically active agent is a beta-blocker, such as propanolol (used, for example, for treating high blood pressure, stage-fright, glaucoma or migraines).

In some embodiments, the pharmaceutically active agent is calcitriol (a vitamin).

In some embodiments, the pharmaceutically active agent methylphenidate (used for example to treat attention deficit disorder).

In some embodiments, the pharmaceutically active agent is testosterone (for hormone replacement therapy).

The pharmaceutically active agent also may be selected from any one or more the following (typical, non-limiting final concentrations in the final composition are indicated as % w/v in parentheses):

In some embodiments, the pharmaceutically active agent is an antibiotic medication, such as clindamycin, tetracycline, gentamicin, metronidizole, bacitracin, neomycin or polymyxin B. In specific embodiments, a composition comprising a combination of antibiotics against different strains of bacteria may be advantageous for certain treatments. A steroidal pharmacological agent, such as betamethasone, in a pharmaceutical composition intended to treat a fungal infection can additionally be included to enhance the retraction of the lesion.

In specific embodiments, the antibiotic medication is clindamycin (1%), tetracycline (1% to 3%), gentamicin (0.1%), metronidizole (0.75%, 1%), bacitracin (250 to 600 units/cc), neomycin (3.5 mg/cc), polymyxin B (8,000 to 12,000 units/cc) or betamethasone (0.025% to 0.1%).

In some embodiments, wound healing can be aided and colonization of wounds (i.e., isolated areas with first-degree burns) can be inhibited by application of a pharmaceutical composition comprising silver sulfadiazine. In specific embodiments, the silver sulfadiazine is at a concentration of 1%. The particular antibiotic selected to be included in the biologic dressing will of course depend on the agents to which the strain of bacteria causing the infection is sensitive, and the specific needs of the patient.

In some embodiments, the pharmaceutically active agent is applied against superficial parasitic infections, such as scabies, nits and lice (including head lice and crab lice) and include miticides or pediculocides such as crotamiton, permethrin, lindane, malathion, benzyl benzoate, thiabendazole and pyrethrins.

In specific embodiments, the active agent is crotamiton (10%), permethrin (5%), lindane (1%), malathion (0.003% to 0.5%), benzyl benzoate (26% to 30%), thiabendazole or pyrethrins.

In some embodiments, the pharmaceutically active agent is an anti-pruritic, such as doxepin, including doxepin (5%), which finds use in relieving the itching in patients with certain types of eczema.

In some embodiments, the pharmaceutically active agent is applied to treat pain associated with arthritis, joint inflammation and muscle pain, such as capsaicin or a corticosteriod. In specific embodiments, the pharmaceutically active agent is menthol (0.40-10%).

In specific embodiments, the corticosteroid are used for treating dermatological inflammatory disorders, such as atopic dermatitis or eczema, seborrheic dermatitis, some forms of psoriasis, aphthous ulcers (canker sores), superficial skin lesions due to contact with poisonous plants such as poison oak or poison ivy, insect bites, and other skin rashes of unknown etiology. Steroidal agents of all different grades (1-7) that include betamethasone, clobetasol, diflorasone, amcinonide, desoximetasone, fluocinonide, halsinonide, triamcinolone, hydrocortisone, flurandrenolide, alclometasone, fluocinolone, desonide, desamethasone, methylprednisolone, clocortolone, fluticasone, mometasone, prednicarbate, amcinonide, and halobetasole.

In specific embodiments, the steroidal agent is betamethasone (0.025% to 0.1%), clobetasol (0.05%), diflorasone (0.05%), amcinonide (0.1%), desoximetasone (0.05% to 0.25%), fluocinonide (0.05%), halsinonide (0.1%), triamcinolone (0.025% to 0.5%), hydrocortisone (0.1% to 2.5%), flurandrenolide (0.05%), alclometasone (0.05%), fluocinolone (0.01% to 0.2%), desonide (0.05%), desamethasone (0.1%) and methylprednisolone (1.0%), clocortolone (0.1%), fluticasone (0.005% to 0.05%), mometasone (0.1%), prednicarbate (0.1%), amcinonide (0.1%) or halobetasole (0.05%).

In some embodiments, the pharmaceutically active agent is a non-steroidal drugs, including salicylic acid and/or retinoic acid (Retin-A), Cantharidin or imiquimod, which are useful for the treatment of warts, including genital warts. In specific embodiments, the pharmaceutically active agent is retin-A.

In specific embodiments, the non-steroidal drug is salicylic acid (0.5% to 60%) and/or retinoic acid (Retin-A) (0.025% to 0.05%), Cantharidin (0.7%) or imiquimod (5%).

In some embodiments, the pharmaceutically active agent is suitable for the treatment of acne, including tretinoin, isotretinoin, adapaline, azelaic acid, clindamycin, erythromycin, tetracycline, benzoyl peroxide or sulfacetamide. In specific embodiments, the agent is tretinoin (0.025% to 0.2%), adapaline (0.1%), azelaic acid (20%), benzoyl peroxide (2.5% to 10%) or sulfacetamide (10%).

In some embodiments, the pharmaceutically active agent is metronidazole, which finds use in the treatment of rosacea. In specific embodiments, the agent is metronidazole (0.75%).

In some embodiments, the pharmaceutically active agent is Anthralin, calcipotriene and/or tazarotene, which find use in the treatment of psoriasis. In specific embodiments, the pharmaceutically active agent is calcipotriene. In specific embodiments, the agent is Anthralin (0.1% to 0.5%), calcipotriene (0.005%) and/or tazarotene.

In some embodiments, the pharmaceutically active agent is an anti-viral agent, such as acyclovir for the treatment of viral infections caused by herpes (type 1 and type 2) simplex viruses or papillomavirus (for example, common and genital warts), gancyclovir, penciclovir, vidarabine, idoxuridine or trifluridine.

In specific embodiments, the anti-viral agent is acyclovir (5%), penciclovir (1%), vidarabine (3%), idoxuridine (0.5%) or trifluridine.

In some embodiments, the pharmaceutically active agent is a local anesthetic, such as lidocaine and other local anesthetics chemically and/or pharmacologically related to lidocaine or lidocaine hydrochloride, such as bupivacaine hydrochloride, etidocaine hydrochloride, mepivacaine hydrochloride, prilocalne hydrochloride, or tetracaine hydrochloride. In specific embodiments, the local anesthetics has low solubility in water, such as benzocaine and the hydroiodide salt of tetracaine.

In specific embodiments, the local anesthetic used in treating mucous membranes and the skin, such as dibucaine, dyclonine hydrochloride, and pramoxine hydrochloride, or capsaicin.

In specific embodiments, the local anesthetic is lidocaine (0.5% to 25%), bupivacaine hydrochloride (0.25% to 1.5%), etidocaine hydrochloride (1.0% to 3.0%), mepivacaine hydrochloride (1.0% to 5.0%), prilocalne hydrochloride (4.0% to 8.0%), tetracaine hydrochloride (0.5% to 2.0%), dyclonine hydrochloride (0.5% to 1.0%), pramoxine hydrochloride (1.0%), or capsaicin (0.025%).

In some embodiments, the pharmaceutically active agent is an antiseptic, such as biguanides such as alexidine, povione iodine, iodine, halogen releasing agents such as iodophor, diamidines, anilides such as triclocarban, phenols, halophenols and bis-phenols such as triclosan, peroxygens such as hydrogen peroxide, silver compounds such as silver nitrate and quarternary ammonium compounds.

In some embodiments, the pharmaceutically active agent is a synthetic hormone, which find use in the treatment of indications associated with abnormal hormone production as well as contraception. For example, a pharmaceutical composition containing transdermal testosterone, generally about 2.5-5.0 mg per application, or equivalent other androgenic compound(s) in an appropriate amount can be used to treat young males with congenital or acquired primary hypogonadism, or congenital or acquired hypogonadotropic hypogonadism and other similar disorders. In women, a pharmaceutical composition containing estradiol (an active form of estrogen) or other equivalent estrogenic compound(s) in an appropriate amount, can be used to treat the indications and symptoms associated with atrophic vaginitis, atrophic dystrophy of the vulva, menopausal symptoms, female hypogonadism, ovariectomy, primary ovarian failure, non-steroid dependent inoperable breast cancer and vasomotor symptoms associated with menopause and prevention of post-menopausal osteoporosis. A pharmaceutical composition containing an estrogenic compound, such as for example estradiol in an amount sufficient for the treatment of such indications is used.

Pharmaceutical compositions comprising norethindrone (progestin) can be used to prevent pregnancy by inhibiting ovulation and thickening the mucosa of the cervix. In addition, a composition containing a progestin compound such as norethindrone, including the agent at 0.14-0.25 mg per application, can be used for treating abnormal menstrual disorders such as amenorrhea, abnormal uterine bleeding and endometriosis, applications generally will be to the skin. The site of application of the composition will vary depending upon the intended use. Generally the site of application will be to the skin at a location that will provide for absorption into the blood stream. Particularly in the case of treatments relating to the female genitalia, application can be intravaginally.

In some embodiments, the pharmaceutically active agent is an agent used for hair growth retardation and stimulation, such as minoxidil, including the minoxidil (1% to 5%).

In some embodiments, the pharmaceutically active agent is an agent used for pigmenting or de-pigmenting the skin, for instance, for use in treating patients with vitiligo, such as hydroquinone or a psoralen agent, such as methoxalen, for combined use with UV light. In specific embodiments, the agent is hydroquinone (2% to 4%) or methoxalen (1.0%).

In some embodiments, the pharmaceutically active agent is an anti-perspirant, anti-anginal, anti-nausea or anti-cancer agent, including, aluminum chloride, for the inhibition of perspiration of isolated dermal areas, for instance to aid in carrying out surgical procedures; nitroglycerin, for the sustained transdermal delivery of this anti-anginal agent; scopolamine, for relief from nausea; and 5-fluorouracil, for the treatment of isolated actinic keratosis lesions. In specific embodiments, the agent is aluminum chloride (20%), nitroglycerin (0.5% to 2.0%) or 5-fluorouracil (5-FU; 5% to 10%).

In some embodiments, as the sole or an additional component, an insect repellant may be included as the pharmaceutically active agent. Examples of insect repellant compounds include terpenoids, such as citronellal, geraniol, terpentine, pennyroyal, cedarwood, eucalyptus and wintergreen; benzoquinones; aromatics, such as cresols, benzaldehyde, cinnamic aldehyde, benzoic acids; and synthetic insect repelling agents, such as N,N-diethyl-m-toluamide (DEET), ethyl hexanediol, dimethyl phthalate, dimethyl ethyl hexanediol, carbate, butopyronoxyl, di-n-propyl isocinchonmeronate, N-octyl bicycloheptene, dicarboximide, and 2,3,4,5-bis(2-butylene)tetra-hydro-2-furaldehyde.

In some embodiments, as the sole or an additional component, a sun protecting, ultraviolet absorptive agents such as a sunscreen, can be used as the pharmaceutically active agent. Such agents include aminobenzoate agents, such as p-aminobenzoic acid (PABA), ethyl 4-[bis(hydroxypropyl)]aminobenzoate, octyl dimethyl PABA, PABA propoxylate, glyceral PABA, 2-ethylhexyl PABA and pentyl PABA; cinnamate agents, such as cinoxate, diethanolamine-p-methoxy cinnamate, 2-ethylhexyl-p-methoxycinnamate and octyl methoxycinnamate; benzones, such as oxybenzone, dioxybenzone, sulisobenzone; salicylates, such as 2-ethylhexyl salicylate, triethanol amine salicylate, and octyl salicylate; and other sunscreen agents, such as meradimate, octinoxate, homosalate, sulisobenzone, titanium dioxide and zinc oxide. In specific embodiments, the agent is meradimate (7.5%), octinoxate (7.5%), homosalate (10%) or sulisobenzone (10%).

In some embodiments, the pharmaceutically active agent is not chlorhexidine gluconate, and the composition does not include chlorhexidine gluconate.

In some embodiments, the pharmaceutically active agent is not nicotine, and the composition does not include nicotine. In specific embodiments, the active agent is scopolamine, while in other specific embodiments the active agent is not scopolamine. In specific embodiments, the active agent is ondansetron, while in other specific embodiments the active agent is not ondansetron. In specific embodiments, the active agent is granisetron, while in other specific embodiments the active agent is not granisetron. In specific embodiments, the active agent is zolmitriptan, while in other specific embodiments the active agent is not zolmitriptan. In specific embodiments, the active agent is dihydroergotamine, while in other specific embodiments the active agent is not dihydroergotamine. In specific embodiments, the active agent is sumatriptan, while in other specific embodiments the active agent is not sumatriptan. In specific embodiments, the active agent is rizatriptan, while in other specific embodiments the active agent is not rizatriptan. In specific embodiments, the active agent is fentanyl, while in other specific embodiments the active agent is not fentanyl. In specific embodiments, the active agent is lidocaine, while in other specific embodiments the active agent is not lidocaine. In specific embodiments, the active agent is cocaine, while in other specific embodiments the active agent is not cocaine. In specific embodiments, the active agent is benzocaine, while in other specific embodiments the active agent is not benzocaine. In specific embodiments, the active agent is alprazolam, while in other specific embodiments the active agent is not alprazolam. In specific embodiments, the active agent is clonazepam, while in other specific embodiments the active agent is not clonazepam. In specific embodiments, the active agent is lorazepam, while in other specific embodiments the active agent is not lorazepam. In specific embodiments, the active agent is diazepam, while in other specific embodiments the active agent is not diazepam. In specific embodiments, the active agent is estazolam, while in other specific embodiments the active agent is not estazolam. In specific embodiments, the active agent is apomorphine, while in other specific embodiments the active agent is not apomorphine. In specific embodiments, the active agent is risperidone, while in other specific embodiments the active agent is not risperidone. In specific embodiments, the active agent is ketorolac, while in other specific embodiments the active agent is not ketorolac. In specific embodiments, the active agent is ibuprofen, while in other specific embodiments the active agent is not ibuprofen. In specific embodiments, the active agent is ketoprofen, while in other specific embodiments the active agent is not ketoprofen. In specific embodiments, the active agent is flurbiprofen, while in other specific embodiments the active agent is not flurbiprofen. In specific embodiments, the active agent is naproxen, while in other specific embodiments the active agent is not naproxen. In specific embodiments, the active agent is astemizole, while in other specific embodiments the active agent is not astemizole. In specific embodiments, the active agent is terfenadine, while in other specific embodiments the active agent is not terfenadine. In specific embodiments, the active agent is cimetidine, while in other specific embodiments the active agent is not cimetidine. In specific embodiments, the active agent is testosterone, while in other specific embodiments the active agent is not testosterone. In specific embodiments, the active agent is retin-A, while in other specific embodiments the active agent is not retin-A.

In some embodiments, the pharmaceutically active agent is mupirocin.

In some embodiments, the pharmaceutically active agent is methyl salicylate, urea (carbamide), camphor, or suboxone.

In general, the pharmaceutically active agent(s) may be present at a concentration typically used for that active agent in a topical formulation, as illustrated above.

In some embodiments, the first composition consists of the pharmaceutically active agent, e.g., the pharmaceutically active agent is combined with the second mixture. In other embodiments, the first composition comprises the pharmaceutically active agent and optionally a first volatile solvent and/or water. In specific embodiments, the pharmaceutically active agent constitutes more than 25% of the first composition. In some embodiments, the pharmaceutically active agent constitutes less than about 25% of the first composition, such as from about 0.1% (w/v) to about 25% (w/v), or from about 0.1% (w/v) to about 20% (w/v), or from about 0.1% (w/v) to about 15% (w/v), or from about 0.1% (w/v) to about 10% (w/v), or from about 0.1% (w/v) to about 5% (w/v), or from about 0.1% (w/v) to about 1% (w/v), or from about 0.5% (w/v) to about 25% (w/v), or from about 0.5% (w/v) to about 20% (w/v), or from about 0.5% (w/v) to about 15% (w/v), or from about 0.5% (w/v) to about 10% (w/v), or from about 0.5% (w/v) to about 5% (w/v), or from about 0.5% (w/v) to about 1% (w/v), or from about 1% (w/v) to about 25% (w/v), or from about 1% (w/v) to about 20% (w/v), or from about 1% (w/v) to about 15% (w/v), or from about 1% (w/v) to about 10% (w/v), or from about 1% (w/v) to about 5% (w/v), or from about 5% (w/v) to about 25% (w/v), or from about 5% (w/v) to about 20% (w/v), or from about 5% (w/v) to about 15% (w/v), or from about 5% (w/v) to about 10% (w/v), or from about 10% (w/v) to about 25% (w/v), or from about 10% (w/v) to about 20% (w/v), or from about 10% (w/v) to about 15% (w/v), or from about 15% (w/v) to about 25% (w/v), or from about 15% (w/v) to about 20% (w/v), or from about 20% (w/v) to about 25% (w/v), including from 0.1-25% (w/v), 0.1-20% (w/v), 0.1-15% (w/v), 0.1-10% (w/v), 0.1-5% (w/v), 0.1-1% (w/v), 0.5-25% (w/v), 0.5-20% (w/v), 0.5-15% (w/v), 0.5-10% (w/v), 0.5-5% (w/v), 0.5-1% (w/v), 1-25% (w/v), 1-20% (w/v), 1-15% (w/v), 1-10% (w/v), 1-5% (w/v), 5-25% (w/v), 5-20% (w/v), 5-15% (w/v), 5-10% (w/v), 10-25% (w/v), 10-20% (w/v), 10-15% (w/v), 15-25% (w/v), 15-20% (w/v) and 20-25% (w/v), including 0.1% (w/v), 0.5% (w/v), 1% (w/v), 5% (w/v), 10% (w/v), 15% (w/v), 20% (w/v or 25% (w/v). However, because the adherent properties of the compositions may provide extended drug exposure and/or controlled drug delivery, in some embodiments a lower concentration can be used while still achieving the desired pharmaceutical effect. In any event, the amount of pharmaceutically active agent can be determined by the skilled artisan.

In some embodiments, such as embodiments directed to transmucosal delivery, the pharmaceutically active agent is present at a dose approved for clinical use and solubilized in a volume of 200 μl or less. In specific embodiments, such as specific embodiments directed to transmucosal delivery, the pharmaceutically active agent is solubilized in a volume of 25-200 μl.

In some embodiments, such as embodiments directed to topical or transdermal delivery, the pharmaceutically active agent is present in a dose approved for clinical use and solubilized in a volume of 2 ml or less. In specific embodiments, such as specific embodiments related to topical or transdermal delivery, the pharmaceutically active agent is solubilized in a volume of 1 ml or less.

Water

In some embodiments, the amount of water (v/v) in the first composition, if present, is from about 1% to about 40%, such as from about 1% to about 30%, from about 1% to about 20%, from about 1% to about 25%, from about 1% to about 10%, from about 1% to about 5%, from about 5% to about 40%, from about 5% to about 30%, from about 5% to about 20%, from about 5% to about 10%, from about 10% to about 40%, from about 10% to about 30%, from about 10% to about 20%, from about 20% to about 40%, from about 20% to about 30%, or from about 30% to about 40% of the first composition, or is about 1%, about 5%, about 10%, about 20%, about 25%, about 30% or about 40% of the first composition, such as the amount of water being from 1-40%, 5-40%, 10-40%, 20-40% or 30-40% of the first composition, including 1%, 5%, 10%, 20%, 25%, 30%, or 40% of the first composition.

In some embodiments, the amount of water (v/v) in the second composition is from about 1% to about 40%, such as from about 1% to about 30%, from about 1% to about 20%, from about 1% to about 25%, from about 1% to about 10%, from about 1% to about 5%, from about 5% to about 40%, from about 5% to about 30%, from about 5% to about 20%, from about 5% to about 25%, from about 5% to about 10%, from about 10% to about 40%, from about 10% to about 30%, from about 10% to about 20%, from about 20% to about 40%, from about 20% to about 30%, or from about 30% to about 40% of the second composition, or is about 1%, about 5%, about 10%, about 20%, about 30% or about 40% of the second composition, such as the amount of water being from 1-40%, 5-40%, 10-40%, 20-40% or 30-40% of the first composition, including 1%, 5%, 10%, 20%, 25%, 30%, or 40% of the second composition. If too much water is present, the resin may lose its biological activity or its characteristic resin properties. In some embodiments where the pharmaceutically active agent is chlorhexidine gluconate, if too much water is present, the resin may lose its ability to improve delivery of chlorhexidine gluconate. On the other hand, if too little water is present, the chlorhexidine gluconate may not go into solution when the first and the second mixtures are combined.

In specific embodiments, the amount of water in the first composition, if any, and the amount in the second composition is selected to provide a final composition comprising water is in a range of 1% to 40% (v/v), including water in a range of 1% to 25% (v/v), including a final amount of water of up to about 25% or up to about 40%, such as up to 25% or up to 40%. If too much water is present, the composition may exhibit instability which may be observed, for example, by the formation of precipitates or the loss of intrinsic properties of the resin. The amount of water that can be present without the formation of unacceptable levels of precipitates may vary with the purity of the resin and/or the identity of the pharmaceutically active agent, and typically ranges from about 1% to about 25% (v/v) water.

As discussed above, in some embodiments, the pharmaceutically active agent is poorly soluble in a typical resin tincture composition comprising volatile solvent. In the context of the methods and compositions described herein, the presence and amount of water in the first composition, if any, and the amount of water in the second composition may be selected and controlled to address this issue. For example, in some embodiments, the presence and amount of water in the first composition, if any, facilitates dissolution of the active agent when mixed with the second composition, which comprises a resin. In other embodiments, the presence and amount of water in the second composition facilitates dissolution of the active agent when mixed with the first composition.

Additionally or alternatively, the presence and amount of water in the final pharmaceutical composition may be selected and controlled to create an environment in which the pH of the final pharmaceutical composition can be adjusted in order to promote a log D of the pharmaceutically active agent that is suitable or advantageous for transdermal or transmucosal delivery.

For example, for transdermal applications, a log D of about 1 or greater may facilitate or enhance transdermal delivery, such as by promoting diffusion through the stratum corneum into the epidermal and dermal layers of the skin. Thus, in some embodiments, such as embodiments for transdermal formulations applied topically to the skin, the presence and amount of water in the final composition may be selected and controlled such that the pH can be adjusted such that the log D of the pharmaceutically active agent is about 1 or greater, about 2 or greater, about 3 or greater, or higher.

The following non-limiting examples are provided to illustrate this aspect of specific embodiments. The pH of the final pharmaceutical composition may be adjusted to about 7.4 for the following pharmaceutically active agents (log D): fentanyl (log D of 3.97); testosterone (log D 3.18); diclofenac (log D of 1.44); tretinoin (i.e., Retin-A) (log D of 4.19).

For transmucosal applications, a log D of about 1 or less may facilitate or enhance transmucosal delivery, such as by promoting diffusion through the mucosal membranes. Thus, in some embodiments, such as embodiments for transmucosal formulations applied to the mucosa, the presence and amount of water in the final composition may be selected and controlled such that the pH can be adjusted such that the log D of the pharmaceutically active agent is about 1 or less, about 0 or less, about −1 or less, about −2 or less, or less.

The following non-limiting examples are provided to illustrate this aspect of specific embodiments. The pH of the final pharmaceutical composition may be adjusted to about 7.4 for the following pharmaceutically active agents (log D): nicotine (log D of −0.62); desloratadine (log D of 0.84); naratriptan (log D of −0.85); ondansetron (log D of 1.2).

These parameters are exemplary, but non-limiting. For example, a composition suitable for transmucosal delivery of vardenafil can be prepared even though the log D of vardenafil is greater than 1. The log D of vardenafil is 3.71 at pH 7.4, but is 2.26 at pH 5.5. Thus, by adjusting the pH of the final pharmaceutical composition to 5.5, the transmucosal delivery of vardenafil may be enhanced. Similarly, while the log D of sildenafil is 2.26 at pH of 7.4, sildenafil has a log D of 1.83 at pH 5.5. Thus, by adjusting the pH of the final pharmaceutical composition to 5.5, the transmucosal delivery of sildenafil may be enhanced.

In some embodiments, the second composition is prepared by combining a resin, a volatile solvent and water, and optionally adjusting the pH of the composition, such as to promote a log D of the pharmaceutically active agent that is suitable or advantageous for transdermal or transmucosal delivery, as discussed above. In some embodiments, the pH of the second composition is adjusted prior to mixing with the first composition. In specific embodiments, the pH of the second composition is adjusted after water is added to the resin, and optionally after the second composition is settled and filtered. In specific embodiments, the resin is mixed with volatile solvent and, subsequently, with increasing amounts of water before the pH is adjusted. The pH of the second composition can be adjusted by methods known in the art, such as by using a base or acid, such as by adding 1-10 N NaOH or 1-6 N HCl.

In some embodiments, the pH of the final pharmaceutical composition is adjusted, such as may be appropriate to promote a log D of the pharmaceutically active agent that is suitable or advantageous for transdermal or transmucosal delivery.

In some embodiments, particulate matter forms after water is added to the resin and volatile solvent mixture. For example, precipitates have been observed to form in benzoin resin tinctures at a pH of about 6.1 or greater. In specific embodiments, such particulate matter may be removed by allowing the second composition to settle, followed by filtering the composition. In specific embodiments, the second composition is allowed to settle for about 1 week, including 1 week, or longer. In other specific embodiments, the second composition is filtered using a 0.45 micron filter. It has been discovered that the settled, filtered second composition exhibits increased stability, including stability to increased pH, and also exhibits increased stability with regard to the pharmaceutically active agent.

Volatile Solvent

As the volatile solvent(s), any pharmaceutically acceptable volatile solvent can be used for the volatile solvent(s) in the first composition, if any, and for the volatile solvent(s) in the second composition, such as ethyl acetate, n-propyl acetate, alcohols such as methanol, ethanol, propanol, and isopropanol, isopropyl alcohol, ketones, such as acetone, and ethers such as dimethyl ether. Other evaporative compounds may also find use, so long as they are compatible with other components of the pharmaceutical compositions and topically acceptable to the majority of patients. In specific embodiments, a volatile solvent in the first composition, if any, and/or a volatile solvent in the second composition is an alcohol such as ethanol, propanol, and isopropanol. In very specific embodiments, a volatile solvent in the first composition and/or a volatile solvent in the second composition is isopropanol. In some embodiments, the volatile solvent(s) in the first composition, if any, is(are) the same as the volatile solvent(s) in the second composition. In other embodiments, the volatile solvent(s) in the first composition, if any, is(are) different from the volatile solvent(s) in the second composition. In other embodiments a volatile solvent among a number of volatile solvents in the first composition, if any, is the same as a volatile solvent among a number of volatile solvents in the second composition. In other embodiments, a volatile solvent in the first composition, if any, and/or a volatile solvent in a second composition is an isopropyl alcohol mixture in water. In specific embodiments, a volatile solvent in the first composition, if any, and/or a volatile solvent in a second composition is a 70% isopropyl alcohol mixture in water.

In some embodiments, the amount of volatile solvent(s) in the second composition is from about 60% to about 99% (v/v), including about 60% to about 95%, or about 60% to about 80%, or 60% to 80%, or 60% to 95%, including 60%, 70%, 80%, 90% 98% and 99% of the total first or second composition. The amount of volatile solvent(s) in first composition, if any, may be similar, but is not particularly limited.

In some embodiments, such as compositions comprising chlorhexidine gluconate, the amount of volatile solvent(s) in the first and second mixtures is independently from about 40% to about 99% (v/v), including about 50% to about 95%, or about 60 to about 99%, or about 60% to about 80%, including 50% to 95%, or 60% to 80%, or 60% to 95%, 60% to 99%, including 40%, 40.2%, 50%, 60%, 70%, 80%, 90% and 99% of the total first or second mixture. In specific embodiments, the amount of volatile solvent in the first mixtures is 70% (v/v). In specific embodiments, the volatile solvent is isopropyl alcohol in an amount of 70% (v/v) in the first mixture.

In specific embodiments, such as compositions comprising chlorhexidine gluconate, the amount of volatile solvent in the first mixtures is 70% (v/v). In specific embodiments, such as compositions comprising chlorhexidine gluconate, the volatile solvent is isopropyl alcohol in an amount of 70% (v/v) in the first mixture.

In some embodiments, the relative amount of the volatile solvent(s) in the first composition, if any, is substantially the same as, or the same as, the relative amount of the volatile solvent(s) in the second composition. In other embodiments, the difference between the relative amount of volatile solvent(s) in the first composition, if any, and second composition is about 25% or less, about 20% or less, about 15% or less, about 5% or less, or about 1% or less, all v/v %. In specific embodiments, the difference between the relative amount of volatile solvent(s) in the first composition, if any, and second composition is 25% or less, 20% or less, 15% or less, 5% or less, or 1% or less, all v/v %. In specific embodiments, the relative amount of the volatile solvent(s) in each of the first composition, if any, and the second composition is independently from about 60% to about 80%, such as from 60% to 80%, all v/v %. For example, the first composition, if any, may comprise 80% volatile solvent while the second composition may comprise 60% volatile solvent (e.g., a difference of 20%). If the difference between the relative amount of solvent(s) in the first and second compositions is too great, precipitation of some pharmaceutically active agents may occur. In specific embodiments, the volatile solvent in both the first (if any) and second compositions is isopropyl alcohol. In other embodiments, the volatile solvent in both the first (if any) and second compositions is isopropyl alcohol, and combining the first and second compositions results in a final concentration of isopropyl alcohol of 70% (v/v).

In specific embodiments, the amount of volatile solvent in the first composition, if any, and second composition is selected to provide a final composition comprising volatile solvent(s) in a range of 60% to 99% (v/v).

Resin

As the resin, any topically and pharmaceutically acceptable resin may be used. For example, suitable resins include, but are not limited to, naturally occurring resins and gums, such as those that are harvested from trees, although gum resins also may be prepared by synthetic means (see for example, U.S. Pat. Nos. 5,644,049, 5,429,590 and 4,307,717). Exemplary resins include benzoin resinous exudate harvested from Styracaceae trees, including Benzoin Siam from Styrax Tonkinesis and Benzoin Sumatra from Styrax Benzoin. Tincture of benzoin and benzoin compound tincture is readily available through numerous commercial sources, including many drug stores and suppliers of surgical goods. Another resinous tree exudate that is commonly used in the medical arts for enhancing the adherence of surgical bandages is mastic, a hard resin that is harvested from Pistacia lentiscus. A tincture of mastic gum (Mastisol) is produced by Ferndale Laboratories in Ferndale, Mich. and is also available through suppliers of surgical goods. Other resins that can be used include the gum resin exudates from Burserceae trees, including Boswellia serrata (also known as Boswellin), Boswellia dalzielli, Boswellia carteri (olibanum gum) and the oleoresin Canarium luzonicum or Canarium commune (Elemi gum or resin). Dammar, olibanum and myrrh gum also can be used in the rein. Oleoresins useful in the compositions of the invention include balsam resins. Additional resinous exudates contemplated from other tree species include Eucalyptus species (Eucalyptus globulus) and Myrtaceae “Tea-tree” species (Melaleuca alternifolia, Leptospermum scoparium, and Kunzea ericoides). Many naturally occurring resins have pharmaceutical properties, and their topical application may cause irritation in certain patients or exacerbate certain conditions. Prudent choice of the resins to be used in preparing a particular pharmaceutical composition takes into consideration the disorder to be treated and the sensitivities of a particular patient's skin. In specific embodiments, the resin is benzoin or mastic gum. In very specific embodiments, the resin is benzoin. In some embodiments, a combination of resins can be used.

In some embodiments, the resin component constitutes more than 20% (w/v) of the second composition. In other embodiments, the resin component constitutes about 20% (w/v) or less of second composition. In specific embodiments, the resin component constitutes from about 1% (w/v) to about 30% (w/v), or from about 1% (w/v) to about 25% (w/v), or from about 1% (w/v) to about 20% (w/v), or from about 1% (w/v) to about 15% (w/v), or from about 1% (w/v) to about 10% (w/v), or from about 1% (w/v) to about 5% (w/v), or from about 2% (w/v) to about 30% (w/v), or from about 2% (w/v) to about 25% (w/v), or from about 2% (w/v) to about 20% (w/v), or from about 2% (w/v) to about 15% (w/v), or from about 2% (w/v) to about 10% (w/v), or from about 2% (w/v) to about 5% (w/v), or from about 3% (w/v) to about 30% (w/v), or from about 3% (w/v) to about 25% (w/v), or from about 3% (w/v) to about 20% (w/v), or from about 3% (w/v) to about 15% (w/v), or from about 3% (w/v) to about 10% (w/v), or from about 3% (w/v) to about 5% (w/v), or from about 5% (w/v) to about 30% (w/v), or from about 5% (w/v) to about 25% (w/v), or from about 5% (w/v) to about 20% (w/v), or from about 5% (w/v) to about 15% (w/v), or from about 5% (w/v) to about 10% (w/v), or from about 10% (w/v) to about 30% (w/v), or from about 10% (w/v) to about 25% (w/v), or from about 10% (w/v) to about 20% (w/v), or from about 10% (w/v) to about 15% (w/v), or from about 15% (w/v) to about 30% (w/v), or from about 15% (w/v) to about 25% (w/v), or from about 15% (w/v) to about 20% (w/v), or from about 16% (w/v) to about 24% (w/v), or from about 16% (w/v) to about 20% (w/v), including from 1-30% (w/v), 1-25% (w/v), 1-20% (w/v), 1-15% (w/v), 1-10% (w/v), 1-5% (w/v), 2-30% (w/v), 2-25% (w/v), 2-20% (w/v), 2-15% (w/v), 2-10% (w/v), 2-5% (w/v), 3-30% (w/v), 3-25% (w/v), 3-20% (w/v), 3-15% (w/v), 3-10% (w/v), 3-5% (w/v), 5-30% (w/v), 5-25% (w/v), 5-20% (w/v), 5-15% (w/v), 5-10% (w/v), 10-30% (w/v), 10-25% (w/v), 10-20% (w/v), 10-15% (w/v), 15-30% (w/v), 15-20% (w/v), 16-24% (w/v) and 16-20% (w/v) of the second composition.

In specific embodiments, the amount of resin in the second composition is selected to provide a final composition comprising resin is in a range of 1% to 25% (w/v). In specific embodiments that may be particularly suitable for preparations to be applied to the skin, the resin is mastic gum and is present in a final concentration in the final composition of from 1.5% to 15% (w/v), including from 1.5% to 5% (w/v), such as from 2.5% to 3.5% (w/v). In specific embodiments that may be particularly suitable for preparations to be applied to the mucosa, the resin is benzoin and is present in a final concentration in the final composition (w/v) of from about 5% to about 35%, such as from about 5% to about 20%, such as from about 5% to about 10%, such as from about 5% to about 8%, such as from about 15% to about 35%, including from 5% to 35%, from 5% to 20%, from 5% to 10%, from 5% to 8%, from 15% to 35%, including about 5%, about 8%, about 10%, about 15%, about 20%, about 35%, including 5%, 8%, 10%, 15%, 20%, and 35%.

Optional Components

The pharmaceutical compositions optionally may include one or more other optional components, which may be provided in the first composition, in the second composition, or which may be added after the first and second compositions are combined.

In some embodiments, the pharmaceutical compositions include a penetration enhancer, i.e., a chemical compound that, when included in a formulation, temporarily increases the permeability of the skin to a drug allowing more of the drug to be absorbed in a shorter period of time. Examples of penetration enhancers that can be used include dimethylsulfoxide (DMSO), n-decyl methyl sulfoxide, N,N-dimethylacetamide, N,N-methyl-2-pyrrolidone and octylphenylpolyethylene glycols.

In some embodiments, menthol and/or peppermint oil may function as enhancers. For example, menthol and/or peppermint oil have been shown to function as enhancers for nicotine, fentanyl, zolmitriptan and dihydroergotamine, when formulated in resin compositions as described herein (e.g., comprising resin and volatile solvent). (Data not shown.) In some embodiments, the method is present at 0.5-20% (w/v). In some embodiments, the peppermint oil is present at 0.5-10% (v/v).

In some embodiments, the pharmaceutical compositions include one or more other pharmaceutically acceptable carriers. Advantageously, any additional carrier does not adversely affect the effectiveness of the pharmaceutically active agent or the resinous delivery vehicle and does not damage the application site. Suitable pharmaceutical carriers include sterile water; saline, dextrose; dextrose in water or saline; condensation products of castor oil and ethylene oxide combining about 30 to about 35 moles of ethylene oxide per mole of castor oil; liquid acid; lower alkanols; oils such as corn oil; olive oil, peanut oil, sesame oil, wintergreen oil, lanolin oil and the like, with emulsifiers such as mono- or di-glyceride of a fatty acid, or a phosphatide, e.g., lecithin, and the like; glycols; polyalkylene glycols; aqueous media in the presence of a suspending agent, for example, sodium carboxymethyl-cellulose; sodium alginate; poly(vinyl pyrrolidone); and the like, alone, or with suitable dispensing agents such as lecithin; polyoxyethylene stearate; and the like.

In some embodiments, suitable pharmaceutical carriers also include glerol and/or glycerin.

Optionally, the composition as a whole or the other carrier may also contain adjuvants such as preserving, stabilizing, wetting, emulsifying agents and the like.

Optionally, the composition may include dyes, fragrances, flavors, and other topically and pharmaceutically acceptable components. In some embodiments, the use of a dye permits easy determination/verification of where the composition has been applied, such as to permit easy determination/verification that the composition has been applied to the entire target site, such as a site being prepared for surgery, or any other site where treatment with the pharmaceutically active agent is desired, such as any other site where the pharmaceutically active agent is being applied to prevent (or reduce the risk of) or treat infection.

Suitable dyes useful for this purpose are known in the art and include triarylmethane dyes (e.g., gentian violet, crystal violet, ethyl violet, and brilliant green), monoazo dyes (e.g., FD&C Yellow No. 5 and FD&C Yellow No. 6), diazo dyes (e.g., D&C Red No. 17), indigoid dyes (e.g., FD&C Blue No. 2), xanthene dyes (e.g., FD&C Red No. 3), anthraquinone dyes (e.g, D&C Green No. 6), quinoline dyes (e.g., D&C Yellow No. 1), and FD&C dyes (e.g., FD&C Blue No. 1 and FD&C Green No. 3). See, e.g., U.S. Pat. No. 8,231,602.

Suitable dyes can also include Cape Aloe.

In some embodiments, the compositions as described herein contain more than one dye. In specific embodiments, the compositions contain FD&C Yellow No. 5, FD&C Yellow No. 6, and FD&C Blue #2. In other embodiments, the compositions contain FD&C Yellow No. 5, and FD&C Yellow No. 6.

In some embodiments, such as where the composition comprises chlorhexidine gluconate, the composition does not include an indigoid dye, such as FD&C Blue No. 2.

In some embodiments, the second composition includes from about 0.1% to about 1% (w/v), or from about 0.1% to about 0.8% (w/v), or from about 0.1% to about 0.5% (w/v) or from about 0.1% to about 0.2% (w/v), or from about 0.2% to about 1% (w/v), or from about 0.2% to about 0.8% (w/v), or from about 0.2% to about to about 0.5% (w/v), or from about 0.5% to about 1% (w/v), or from about 0.5% to about 0.8% (w/v), or from about 0.8% to about 1% (w/v) of the dye, including 0.1-1% (w/v), 0.1-0.8% (w/v), 0.1-0.5% (w/v), 0.1-0.2% (w/v), 0.2-1% (w/v), 0.2-0.8% (w/v), 0.2-0.5% (w/v), 0.5-1% (w/v), 0.5-0.8% (w/v) and 0.8-1% (w/v) of the dye, such as 0.1% (w/v), 0.2% (w/v), 0.5% (w/v), 0.8% (w/v) and 1% (w/v) of the dye.

In some embodiments, it has been found that the presence of an anionic dye (or other anionic species) in a composition comprising a resin tincture and chlorhexidine gluconate leads to the formation of a complex between the anion and chlorhexidine gluconate, which in turn leads to precipitation and other stability problems, such as inactivation of the chlorhexidine gluconate. It has been surprisingly discovered that this problem can be minimized or avoided by preparing the dye in a solution comprising 15-45% water (v/v), adding that dye solution to the resin tincture, and then allowing the solution to settle prior to mixing with chlorhexidine gluconate. For example, the dye can be dissolved in water and then combined with a volatile solvent (such as any described above) to prepare a composition comprising about 55-85% (v/v) volatile solvent and about 15-45% water, such as 15%, 20%, 25%, 30%, 40% or 45% water. Then, the dye composition can be added to the second composition (containing resin) before it is added to the first composition. Alternatively, the dye composition can be added after the first and second compositions are combined.

Additionally or alternatively, the stability and/or antibacterial property of chlorhexidine gluconate in the pharmaceutical composition can be promoted by providing an optimal pH environment. The complex formation and subsequent stability problems described above typically occur at high pH levels. On the other hand, having too low of a pH in the pharmaceutical composition can cause the composition to be physically irritating to the surface upon which the composition is topically applied. Thus, the pH level of the pharmaceutical composition may be controlled to create an environment that promotes the stability of chlorhexidine gluconate without causing irritation. In some embodiments, the pH level is controlled with the addition of water. Without being bound by theory, it is believed that water causes the esters in some resins, such as benzoin, to hydrolyze and form acids that prevent chlorhexidine gluconate from complexing with any dye in the composition. Additionally or alternatively, the second mixture can be treated with a base or acid, such as NaOH or HCl, to achieve an optimal pH to maximize the anti-bacterial property of chlorhexidine gluconate. In specific embodiments, the pH is from about 5.5 to about 7.0, including from about 6.0 to about 7.0, including from 5.5 to 7.0, including from 6.0 to 7.0. In other specific embodiments, the final pH of the pharmaceutical composition is from about 5.5 to about 7.0, including from about 6.0 to about 7.0, including from 5.5 to 7.0, including from 6.0 to 7.0. In other specific embodiments, the base or acid concentration is 1-10 N. In other specific embodiments, the base or acid is added to the second mixture after the dye is added to the second mixture.

The relative amounts of the components can be varied from and within the above ranges in order to obtain a composition with desired pharmaceutical strength, desired pharmacokinetic properties, and/or desired physical properties, which may depend on the site of application (e.g., skin versus nail versus mucosa). For example, the relative amounts may affect the rate of release of the chlorhexidine gluconate, the tackiness of the film, and/or the thickness or area of application. For example, a composition formulated for application to the face may have a lower relative amount of resin to provide a less sticky composition and a thinner film upon application and evaporation of the volatile solvent(s).

In some embodiments, chlorhexidine gluconate pharmaceutical compositions can be prepared as a sticky slurry or solution, which can be applied to a site on the skin, nail or a mucosal membrane, such as the buccal mucosal membrane. The consistency of the pharmaceutical composition can be varied by, for example, adjusting the relative amount of volatile solvent and resin. For areas where evaporation of the volatile solvent may be slower, such as mucosal membrane such as the gums, a composition with less volatile solvent may be advantageous. On the other hand, for areas that are hard to reach, such as between the toes, a composition with more volatile solvent may be advantageous. Still, for treatment requiring a more prolonged effect, a more viscous composition (with less volatile solvent) may be advantageous.

Methods of Using Pharmaceutical Compositions

Pharmaceutical compositions prepared by the methods described herein can be applied to the skin, nail or mucosa to achieve topical, transdermal or transmucosal delivery of the pharmaceutically active agent. In some embodiments, the compositions described herein form a film on the site of application, after application and evaporation of the volatile solvent(s). In specific embodiments, the film is a hydrophobic film. Generally, the film comprises the resin and pharmaceutically active agent, and retains the pharmaceutically active agent in contact with the application site, e.g., the skin, nail or mucosa, providing sustained delivery of the active agent. The film also may provide a protective coating for the active agent.

The pharmaceutical composition may initially be prepared in any form suitable for topical (e.g., transdermal or mucosal) application, such as a paste, a liquid, a semi-solid, a gel, a suspension, an emulsion or the like. To minimize waste, application is generally carried out by painting, swabbing or placing one or more drops of the composition at the affected site or sites, but certain preparations can also be applied by spraying on the formulation, and allowing it to dry.

The pharmaceutical compositions can be applied to any suitable site, such as a site of a superficial skin lesion or infection, such as on cutaneous areas, fingernails, toenails, mucous membranes, and mucocutaneous junctions (i.e., perianal, intertriginous and vulvovaginal areas), or any site for systemic effect.

In one embodiment, a drop of the composition can be applied to any suitable site such as any mucosal site, such as the inside of the cheek. This embodiment may be particularly advantageous when the pharmaceutically active agent exhibits systemic activity via transdermal or transmucosal delivery.

In some embodiments, the composition is applied to a surgical site, prior to or after surgery, to provide sustained release of an anti-infection agent, such as antiseptics. For example, a composition comprising bacitracin as the pharmaceutically active agent can be applied to a surgical site prior to or after surgery. In another example, a composition comprising mupirocin as the pharmaceutically active agent can be applied to a surgical site prior to or after surgery. In another example, a composition comprising chlorhexidine gluconate can be applied to a surgical site prior to or after surgery. In specific embodiments, a composition comprising chlorhexidine gluconate is applied to a surgical site prior to surgery. In other embodiments, a composition comprising chlorhexidine gluconate is applied to a skin, nail or mucosal site at risk of infection.

Depending on the condition being treated, the composition may be applied once a day, twice a day, or more or less frequently.

The composition conveniently can be removed at will, by application of an appropriate solvent, normally ethanol. The composition can also be removed by scrubbing with soap and water.

In some embodiments, the film formed on the application site by the pharmaceutical compositions upon application and evaporation of the solvent(s) are non-occlusive but adherent on the application site (e.g., skin or nail or mucosa). In other embodiments, the film serves as a protective coating at the application site. Additionally or alternatively, the film may act as a reservoir for the pharmaceutically active agent to provide sustained delivery of pharmaceutically active agent.

In some embodiments, the pharmaceutical composition persists at the site of application for greater than four hours, including as long as 8, 10 or 12 hours or longer, such as 16, 18 or 20 hours or longer, including as long as 24, 36 or 72 hours, or even longer. The duration can be selected based on the pharmaceutically active agent, the condition being treated, etc.

Applicator for Pharmaceutical Compositions

In some embodiments, a composition as described herein is provided in an applicator. In some embodiments, the applicator is a dual-chamber applicator, with a first chamber containing the first composition and the second chamber containing the second composition. A wall between the first chamber and second chamber may be opened or broken prior to use, to permit combining of the first composition or second composition. Additionally or alternatively, the applicator may include a third chamber which can receive each of the first composition and the second composition to combine them prior to use. In some embodiments, the first and second compositions are mixed together for about one minute. In other embodiments, the first and second compositions are mixed together for about less than one minute, such as from about 10 to about 60 seconds, or from about 10 to about 50 seconds, or from about 10 to about 40 seconds, or from about 10 to about 30 seconds, or from about 10 to about 20 seconds, or from about 15 to about 60 seconds, or from about 15 to about 50 seconds, or from about 15 to about 40 seconds, or from about 15 to about 30 seconds, or from about 15 to about 20 seconds, or from about 20 to about 60 seconds, or from about 20 to about 50 seconds, or from about 20 to about 40 seconds, or from about 20 to about 30 seconds, or from about 40 to about 60 seconds, or from about 30 to about 50 seconds, or from about 30 to about 40 seconds, or from about 40 to about 60 seconds, or from about 40 to about 50 seconds, or from about 50 to about 60 seconds, including from 10-60 seconds, 10-50 seconds, 10-40 seconds, 10-30 seconds, 10-20 seconds, 15-60 seconds, 15-50 seconds, 15-40 seconds, 15-30 seconds, 15-20 seconds, 20-60 seconds, 20-50 seconds, 20-40 seconds, 20-30 seconds, 30-60 seconds, 30-50 seconds, 30-40 seconds, 40-60 seconds, 40-50 seconds and 50-60 seconds. In specific embodiments, the first and second compositions are mixed together for one minute or 10, 15, 20, 30, 40 or 50 seconds.

In other embodiments, the applicator contains a composition obtained by combining the first composition and the second composition. For example, the applicator may be a single chamber applicator.

The compositions described herein can be provided in one or more applicators.

Additionally or alternatively, the compositions can be provided in kits with one or more doses. In some embodiments, the kits include containers (which also can serve as an applicator), which contains a ready-to-use composition, or a composition (including a lyophilized composition), to be diluted prior to use. The containers can be made of plastic, glass, metal or such material deemed appropriate for each particular medication and can be light opaque as required for light sensitive formulations. The containers can be color-coded, each color being unique to a particular product and its respective active ingredient. The containers can also be color coordinated with the outer packaging to simplify marketing and consumer purchasing.

Examples of containers that are also applicators are those that facilitate application of the subject compositions to the skin or mucosa, such as bottles (optionally with a brush or sponge to facilitate application), roll-on and spray applicators (including manual and aerosolized), applicators with small padded applicator tips for the delivery of buccal mucosal medications, or syringe type applicators for semisolid medication such as are described in U.S. Pat. No. 5,531,703 and references cited therein, particularly for the delivery of vaginal mucosal medications.

The pharmaceutical compositions can be contained in packaging material which comprises a label indicating that the compositions can be used to treat dermatologic disorders in humans or to treat other disorders in humans using transdermal or transmucosal delivery means, or to provide anti-infection activity, such as may be needed or desired at a surgical site or other site at risk of infection.

The invention now being generally described, it will be more readily understood by reference to the following examples which are included for purposes of illustration only and are not intended to limit the present invention.

### Example 1

### First Composition

A first composition is prepared that consists of pharmaceutically active agent (alone), or that comprises pharmaceutically active agent and optionally a first volatile solvent and/or water

### Second Composition

A second composition is prepared by combining adding a resin and a volatile solvent to a mixing vessel and stirring the mixture for about several hours or overnight. If not stirred overnight, the stirred time can be from about 3-6 hours, 3-5 hours, 3-4 hours, or 4 hours. After settling for about an hour, the resulting solution is decanted into another vessel. The settling time may be about 1 hour.

The supernatant is decanted or the particulate removed using a cheesecloth. The supernatant is then filtered twice, first at 5 microns and then at 0.45 microns. The filtering can be performed using a syringe filter, including a syringe nylon filter. The amount of volatile solvent lost due to evaporation while settling is calculated, and this amount is added to the solution in the vessel.

Water is added to the filtered solution. The mixture is then filtered, which can be accomplished using a syringe filter, including a syringe nylon filter, a 5-micron syringe nylon filter and/or a 0.45-micron syringe nylon filter. If too much water is added, the adhesive characteristics and biological activity of the resin can be diminished after the solution is filtered.

Alternatively, to prepare the second composition, resin is mixed with isopropal alcohol and water, followed by filtering sequentially at 5 microns and then at 0.45 microns, which can be accomplished using a syringe filter, including a syringe nylon filter.

Depending on the active agent (such as for nicotine, diclofenac, ondansetron or sildenafil), the pH of the second composition is raised to greater than 6.1 before the first and second compositions are combined, such as by adding 1-10 N NaOH. If a precipitate forms at about pH 6.1, it can be removed by letting the second composition settle and then filtering. For example, the second composition may be allowed to settle for a period of time such as one week, and then the resulting supernatant may be filtered, such as using a 0.45 micron filter. Then the pH may be adjusted (increased) further as desired.

Optionally, dye and water are separately mixed until the dye is dissolved, and volatile solvent is added and mixed to produce a dye solution. The dye solution and the second composition are combined with water and mixed overnight. The dye/second composition is filtered, such as on a large or small scale. The filter can be a syringe filter, including a syringe nylon filter, a 5-micron syringe nylon filter or a 0.45-micron syringe nylon filter.

Equal volumes of the first composition and second composition are combined by simple mixing over a period of about 15 to about 30 seconds.

### Example 2

Chlorhexidine gluconate and a first volatile solvent are added to a first mixing vessel and stirred to produce a first mixture.

A resin and a volatile solvent are added to a second mixing vessel and stirred for about several hours to produce a solution. The stirred time can be from about 3-6 hours, 3-5 hours, 3-4 hours, or 4 hours. After settling for at least an hour, the solution is decanted into another vessel. The settling time may be about 1 hour, several hours or overnight. The amount of volatile solvent lost due to evaporation while settling is calculated, and this amount is added to the solution in the vessel. Water is added and the resulting (second) mixture is filtered to produce a tincture. The filter can be a syringe filter, including a syringe nylon filter, a 5-micron syringe nylon filter, a 0.45-micron syringe nylon filter or any filter used in large scale mass production. This mixture is allowed to settle for a period, usually 12-48 hours, to allow the supernatant to completely clarify. At this point, there is a clear partition between the supernatant and an underlying, denser, organic liquid phase. The supernatant is then carefully decanted or otherwise separated from the underlying liquid so that the liquid does not mix with the supernatant.

Dye and water are separately mixed until the dye is dissolved, and volatile solvent is added and mixed to produce a dye solution comprising 15-45% (v/v) water. The dye solution and the tincture solution are combined with water and mixed overnight. The dye/tincture mixture is filtered and allowed to settle. The filter can be a syringe filter, including a syringe nylon filter, a 5-micron syringe nylon filter, a 0.45-micron syringe nylon filter or any filter used in large scale mass production. After settling, the pH of the dye/tincture mixture may adjusted, such as by using 1-10 N NaOH or HCl, to achieve a pH that supports the antimicrobial activity of chlorhexidine gluconate, such as a pH of from about 5.5 to about 7.0, including from about 6.0 to about 7.0, including from 5.5 to 7.0, including from 6.0 to 7.0.

Equal volumes of the first mixture and second mixture are combined by simple mixing over a period of about 15 to about 60 seconds. The final composition comprises up to 25-40% (v/v) water.

### Example 3

Chlorhexidine gluconate is commercially available from Xttrium Laboratories (Mt. Prospect, Ill.) in a 20% (w/v) solution in water (20 mL of the solution contains 4 g of chlorhexidine gluconate).

A first mixture is prepared by adding 20 mL of the 20% chlorhexidine gluconate solution to 10 mL of water and 70 mL of 100% isopropyl alcohol, resulting in a chlorhexidine gluconate concentration of 4% (w/v) in 70% (v/v) isopropyl alcohol.

A second mixture is prepared by adding 160 g benzoin to 497.5 mL isopropyl alcohol and stirring overnight. After settling for about an hour in the morning, the supernatant is removed either by using a cheesecloth or by decanting. The supernatant is filtered twice, first at 5 microns and then at 0.45 microns. The filtering can be performed, such as on a large or small scale, using a syringe filter, including a syringe nylon filter. Isopropyl alcohol is added to the filtered solution to make up for the amount of volatile solvent lost due to evaporation while settling.

213.5 mL of deionized water is added to the filtered solution and the mixture is stirred for 1 hour, and the emulsion is left to settle overnight. The solution is allowed to settle until the supernatant clarifies and there is a clear partition between the overlying supernatant and the underlying, denser, organic liquid phase. The partitioned (water-containing) supernatant is decanted and filtered, such as with a syringe filter, including a syringe nylon filter, a 5-micron syringe nylon filter and/or a 0.45-micron syringe nylon filter.

Alternatively, the second mixture is prepared by mixing benzoin with isopropyl alcohol and water, followed by filtering the resulting mixture sequentially at 5 microns and then at 0.45 microns, which can be accomplished using a syringe filter, including a syringe nylon filter.

The filtered solution is brought up to 711 mL with 70% (v/v) isopropyl alcohol, bringing the content of benzoin to 22.5% (w/v).

A dye solution is produced by mixing 4.688 g. FD&C Yellow #5, 2.839 g. FD&C #6 and 0.516 g. FD&C Blue #2 for a total of 8 g. The mixture is then combined and mixed with 85.2 ml of water until the dyes are solubilized. The solubilized mixture is then mixed with 198.8 ml of isopropyl alcohol, followed by filtering the resulting mixture sequentially at 5 microns and then at 0.45 microns. The filtering can be accomplished using a syringe filter, including a syringe nylon filter.

Alternatively, 284 mL of 70% (w/v) isopropyl alcohol is added to the 8 g dye mixture and stirred for 1 hour. The stirred dye mixture is left to settle overnight, followed by decanting of the supernatant and filtering of the supernatant sequentially at 10 microns and 0.45 microns. The filtered dye solution is brought up to 284 mL with 70% (w/v) isopropyl alcohol, bringing the content of the dye to 2.8% w/v in the alcohol.

Alternatively, the dye solution is then mixed with the second mixture overnight and subsequently filtered at 0.45 microns and brought up to 1 L with 70% (v/v) isopropyl alcohol.

The dye/second mixture composition is stored at room temperature, 30° C. or 40° C. in containers to prevent evaporation and checked monthly for precipitation or other evidence of instability.

To form the final pharmaceutical composition, the dye/second mixture composition is allowed to settle for 1 month at room temperature, and its pH is adjusted to a pH of 6.5 using 10 N NaOH. This mixture is combined with the first mixture in equal volumes by simple mixing over a period of about 15 to about 30 seconds.

### Example 4

A first mixture is prepared by adding chlorhexidine gluconate to isopropyl alcohol (IPA) such that the mixture is 100 mL with 4% chlorhexidine gluconate in 70% (v/v) IPA at pH 6.5.

A second mixture is prepared by adding 800 grams of benzoin resin to 1.972 L of 100% IPA and filtered. 100% IPA is then added to the filtered solution make up for loss of volume during the filtering.

845 mL of deionized water is added to the filtered solution and subsequently filtered with a 0.45 micron filter. 70% (v/v) IPA is then added to make up for loss of volume during the filtering.

The pH of the filtered solution is brought to between about 6.0 and 7.0 by adding 10N NaOH, in order to provide for a final benzoin concentration of 5% (w/v) or 8% (w/v). One or both of 100% and 70% (v/v) IPA is added to the pH-adjusted solution to provide a final benzoin mixture.

Taking 71.6 mL of the benzoin mixture, add 21.3 mL of dye (prepared by mixing 4.688 grams FD&C Yellow No. 5 and 0.284 grams FD&C Yellow No. 6; adding 70% IPA to reach a volume of 284 mL; filtering the mixture at 0.45 microns; and bringing the volume back up to 284 mL with 70% IPA) and 7.1 mL of 70% (v/v) IPA.

The benzoin/dye mixture is refrigerated overnight. The resulting supernatant is then decanted while cold and 70% (v/v) IPA is added to the supernatant to bring the volume up to 100 mL to arrive at the second mixture. The second mixture is allowed to stand overnight at room temperature to equilibrate.

The first and second mixtures are combined and stored in a closed container in the dark at room temperature until use.

### Example 5

A first mixture is prepared with the following components:

60%-80% (v/v) isopropyl alcohol;
20%-40% (v/v) water; and
4% (w/v) chlorhexidine gluconate.

A second mixture is prepared with the following components:

60%-80% (v/v) isopropyl alcohol;
20%-40% (v/v) water;
16%-20% (w/v) resin; and
0.2%-0.8% (w/v) dye.

The first mixture is combined with the second mixture to obtain a pharmaceutical composition.

### Example 6

A study was conducted to confirm the stability of chlorhexidine gluconate in a resin composition as described herein. The composition contained 2% (w/v) chlorhexidine gluconate, 8% (w/v) benzoin gum, 0.8% (w/v) dye, 10% (v/v) water and 70% (v/v) isopropyl alcohol. Specifically, the degradation of chlorhexidine gluconate into 4-chloroaniline, a well-characterized toxic substance that can cause hemolysis and methemoglonemia, was assessed when the composition was stored at 40° C. The degradation was determined to be less than 1% over 5 months, demonstrating the stability of the compositions described herein.

Although the invention has been described with reference to the above examples, it will be understood that modifications and variations are encompassed within the spirit and scope of the invention. Accordingly, the invention is limited only by the following claims.

### Example 7

Pharmaceutical compositions comprising vardenafil or sildenafil mesylate were prepared along the lines described in Example 1, with the following components:

Vardenafil (free base) (10 mg/75 μL) at 13.3% (w/v); 20% (w/v) benzoin gum in 79% (w/v) ETOH/21% (w/v) water; 2% (v/v) peppermint oil; pH=4.1.
Sildenafil Mesylate: 25 mg/75 μL at 25% (w/v); 20% (w/v) benzoin gum in 79% (w/v) ETOH/21% (w/v) water; 5%(w/v) L-methanol; 2% (w/v) peppermint oil; 0.1% (v/v) sucralose; pH=5.2.

### Example 8

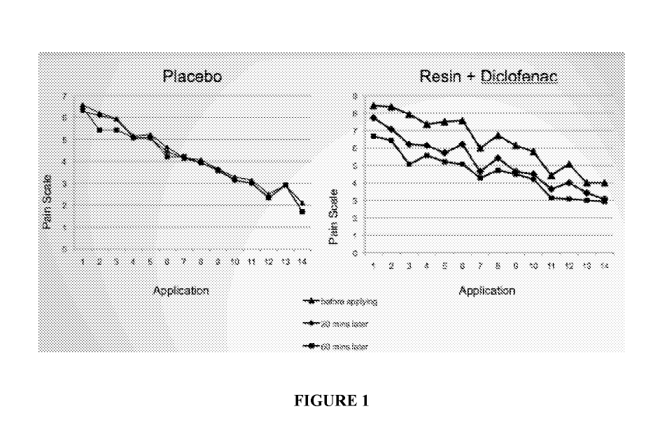

A clinical study was conducted to determine the effect of a diclofenac resin composition as described herein on pain, when the composition is administered topically to an individual. The composition contained 2% (w/v) of diclofenac, 5% (w/v) menthol, 3.5% (w/v) mastic gum, 70% (v/v) isopropyl alcohol, and 30% (v/v) water, at a pH of 8.0. The composition was administered twice per day to patients suffering from acute injuries received in the previous 48 hours. Pain was assessed prior to application and 20 and 60 minutes after application, according to an Analogue Pain Scale Score. FIG. 1 depicts the profile of pain level before treatment and 20 and 60 minutes after treatment. The data presented in the figure demonstrate the efficacy of the diclofenac resin composition, when compared to a placebo.

### Example 9

In vitro studies were performed to determine the in vitro diffusion profiles of diclofenac, calcipotriene and Retin-A from resin compositions as described herein.

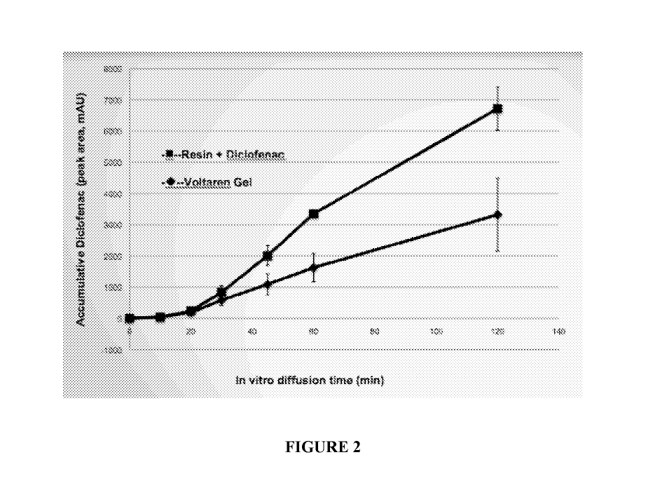

FIG. 2 shows the in vitro accumulation profile of a diclofenac resin composition as compared to Voltaren® 1% diclofenac gel. The resin composition contained 2% (w/v) of diclofenac, 5% (w/v) menthol, 3.5% (w/v) mastic gum, 70% (v/v) isopropyl alcohol, and 30% (v/v) water, at a pH of 8.0. The data presented in the figure demonstrate that the diclofenac resin composition achieves higher and more sustained delivery of diclofenac.

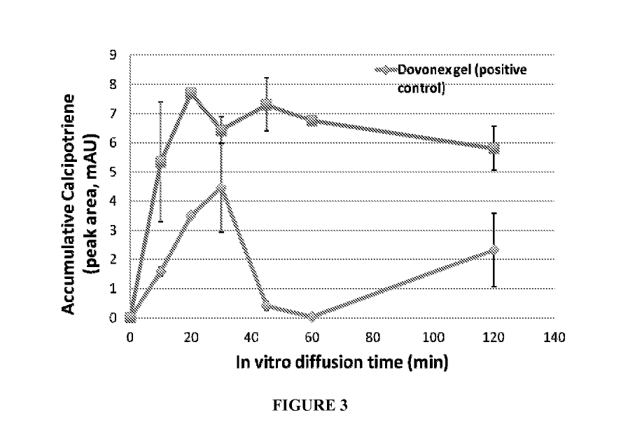

FIG. 3 shows the in vitro accumulation profile of a calcipotriene resin composition as compared to Dovonex®, a commercially available calcipotriene cream. The resin compositions contained 0.005% (w/v) calcipotriene, 3.5% (w/v) mastic gum, 70% (v/v) isopropyl alcohol, and 30% (v/v) water, at a pH of 8.0. The Dovonex® cream contained 0.005% (w/v) calcipotriene. The data presented in the figure demonstrate that the calcipotriene resin composition achieves higher, more rapid and more sustained delivery of calcipotriene across human epidermal cells.

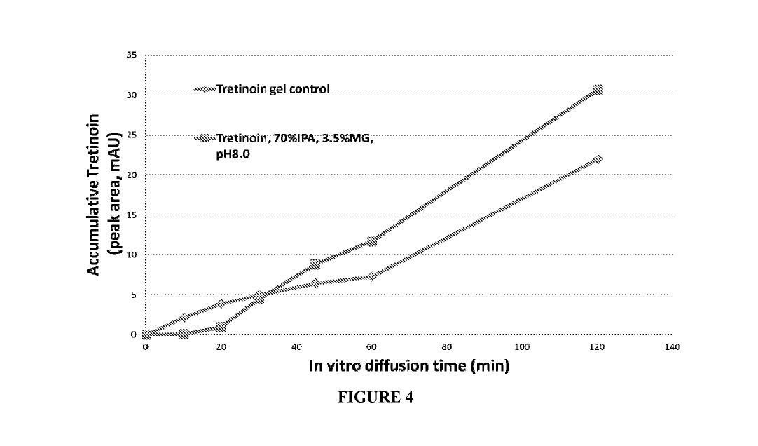

FIG. 4 shows the in vitro accumulation profile of a Retin-A resin composition as compared to a commercial Retin-A cream. The resin composition contained 0.025% (w/v) Retin-A, 3.5% (w/v) mastic gum, 70% (v/v) isopropyl alcohol, and 30% (v/v) water, at a pH of 8.0.

The commercial Retin-A cream also contained 0.025% retin-A. The data presented in the figure demonstrate that the resin composition achieves higher, more rapid delivery of Retin-A.

1.-52. (canceled)

53. A method for producing a chlorhexidine gluconate pharmaceutical composition for topical delivery, comprising combining:
(i) a first composition comprising chlorhexidine gluconate, a first volatile solvent, and 1-40% (v/v) water; and
(ii) a second composition comprising a resin, a second volatile solvent, and 1-40% (v/v) water,
wherein the final amount of water in the chlorhexidine gluconate pharmaceutical composition is up to 40% (v/v).

54. A method of preparing a pharmaceutical composition for topical, transdermal or transmucosal delivery of a pharmaceutically active agent, comprising combining:
(i) a first composition comprising or consisting of a pharmaceutically active agent and optionally a first volatile solvent and/or water; and
(ii) a second composition comprising a resin, a second volatile solvent, and water.

55. The method of claim 54, wherein the active agent is selected from the group consisting of nicotine, scopolamine, lidocaine, benzocaine, ketorolac, ibuprofen, ketoprofen, flurbiprofen, naproxen, astemizole, terfenadine, cimetidine, testosterone, retin-A.

56. The method of claim 54, wherein the active agent is selected from the group consisting of ondansetron, granisetron, zolmitriptan, dihydroergotamine, sumatriptan, rizatriptan, fentanyl, cocaine, alprazolam, clonazepam, lorazepam, diazepam, estazolam, apomorphine, risperidone, buprenorphine, naloxone, flumazenil, tadalafil, vardenafil, sildenafil, sildenafil mesylate, dolasetron, palonsetron, triazolam, naratriptan, diclofenac, etololac, meclofenamate, indocin, meloxicam, nabumetone, oxaprozin, prioxicam, sulindac, tolmetin, celecoxib, loratadine, desloratidine, cetirizine, morphine, hydromorphine, levorphanol, meperidine, oxycodone, oxymorphone, propanolol, calcitriol, and methylphenidate.

57. The method of claim 54, wherein the resin is benzoin or mastic gum.

58. The method of claim 53, wherein the resin is benzoin or mastic gum.

59. The method of claim 53, wherein at least one of the first composition and the second composition is filtered prior to combining with the other composition.

60. The method of claim 53, wherein the first and/or the second volatile solvents are independently selected from the group consisting of ethyl acetate, n-propyl acetate, methanol, ethanol, propanol, isopropanol, isopropyl alcohol, acetone and dimethyl ether.

61. The method of claim 53, further comprising preparing the second composition by combining the resin, volatile solvent and water, allowing the composition to settle, and filtering off any particulate matter.

62. The method of claim 53, wherein the pH of the second composition is adjusted to adjust the log D of the pharmaceutically active agent to enhance transdermal or transmucosal delivery.

63. The method of claim 53, wherein
(i) the first composition comprises 60%-80% (v/v) isopropyl alcohol, 20%-40% (v/v) water, and 4% (w/v) chlorhexidine gluconate; and
(ii) the second composition comprises 60%-80% (v/v) isopropyl alcohol, 20%-40% (v/v) water, and 16%-20% (w/v) resin.

64. A pharmaceutical composition made by the method of claim 53.

65. A pharmaceutical composition made by the method of claim 54.

66. A pharmaceutical composition comprising a pharmaceutically active agent, a volatile solvent, a resin, and 10-40% (v/v) water.

67. The composition of claim 66, wherein the resin is benzoin or mastic gum.

68. The composition of claim 66, wherein the pharmaceutically active agent is chlorhexidine gluconate.

69. The composition of claim 66, wherein the pharmaceutically active agent is selected from the group consisting of nicotine, scopolamine, lidocaine, benzocaine, ketorolac, ibuprofen, ketoprofen, flurbiprofen, naproxen, astemizole, terfenadine, cimetidine, testosterone, retin-A.

70. The composition of claim 66, wherein the active agent is selected from the group consisting of ondansetron, granisetron, zolmitriptan, dihydroergotamine, sumatriptan, rizatriptan, fentanyl, cocaine, alprazolam, clonazepam, lorazepam, diazepam, estazolam, apomorphine, risperidone, buprenorphine, naloxone, flumazenil, tadalafil, vardenafil, sildenafil, sildenafil mesylate, dolasetron, palonsetron, triazolam, naratriptan, diclofenac, etololac, meclofenamate, indocin, meloxicam, nabumetone, oxaprozin, prioxicam, sulindac, tolmetin, celecoxib, loratadine, desloratidine, cetirizine, morphine, hydromorphine, levorphanol, meperidine, oxycodone, oxymorphone, propanolol, calcitriol, and methylphenidate.

71. A method of topically, transdermally or transmucosally administering a pharmaceutical composition to an individual, comprising applying a pharmaceutical composition according to claim 66 to the skin, nail or mucosa of the individual.

72. A container containing a pharmaceutical composition according to claim 66 in a single chamber.

73. A container comprising:
(i) a first chamber comprising a first composition comprising or consisting of a pharmaceutically active agent and optionally a first volatile solvent and/or water and
(ii) a second chamber comprising a second composition comprising a resin, a second volatile solvent, and water.
